(19) United States
(12) Patent Application Publication
(10) Pub. No.: US 2013/0172297 A1
(43) Pub. Date: Jul. 4, 2013

(54) Treatment of Autoimmune Diseases

(71) Applicants: Peter Gergely, Oberwil (CH); Erik Wallstroem, Muttenz (CH)

(72) Inventors: Peter Gergely, Oberwil (CH); Erik Wallstroem, Muttenz (CH)

(73) Assignee: NOVARTIS AG, Basel (CH)

(21) Appl. No.: 13/643,320

(22) PCT Filed: May 5, 2011
(86) PCT No.: PCT/EP11/57203

§ 371 Date: Jan. 8, 2013

(30) Foreign Application Priority Data
May 6, 2010 (EP) 10162079.7

(51) Int. Cl. A61K 31/661; A61K 31/137

(52) U.S. Cl. CPC A61K 31/661; A61K 31/137
USPC 514/114; 514/653

(57) ABSTRACT

Disclosed is the use of a compound of formula I wherein X is O, S, SO or SO₂; R₁ is halogen, trihalomethyl, —OH, C₁₋₇alkyl, C₁₋₄alkoxy, trifluoromethoxy, phenoxy, cyclohexylmethyloxy, pyridylmethoxy, cinnamyloxy, naphthylmethoxy, phenoxymethyl, —CH₂—OH, —CH₂—CH₂—OH, C₁₋₄alkylthio, C₁₋₄alkyl-sulfinyl, C₁₋₄alkylsulfonyl, benzylthio, acetyl, nitro or cyano, or phenyl, phenylC₁₋₄alkyl or phenyl-C₁₋₄alkoxy each phenyl group thereof being optionally substituted by halogen, CF₃, C₁₋₄alkyl or C₁₋₄alkoxy; R₂ is H, halogen, trihalomethyl, C₁₋₄alkoxy, C₁₋₇alkyl, phenethyl or benzyloxy; R₃H, halogen, CF₃, OH, C₁₋₇alkyl, C₁₋₄alkoxy, benzyloxy, phenyl or C₁₋₄alkoxymethyl; each of R₄ and R₅, independently is H or a residue of formula (a) wherein each of R₈ and R₉, independently, is H or C₁₋₄alkyl optionally substituted by halogen; and n is an integer from 1 to 4; or a pharmaceutically acceptable salt, hydrate, solvate, isomer or prodrug thereof; or a compound of formula II wherein R₁ₐ is halogen, trihalomethyl, C₁₋₄alkyl, C₁₋₄alkoxy, C₁₋₄alkylthio, C₁₋₄alkylsulifinyl, C₁₋₄alkylsulfonyl, aralkyl, optionally substituted phenoxy or aralkyloxy; R₂ₐ is H, halogen, trihalomethyl, C₁₋₄alkyl, C₁₋₄alkoxy, aralkyl or aralkyloxy; R₃ₐ is H, halogen, CF₃, C₁₋₄alkyl, C₁₋₄alkoxy, C₁₋₄alkylthio or benzyloxy; R₄ₐ is H, C₁₋₄alkyl, phenyl, optionally substituted benzyl or benzoyl, or lower aliphatic C₁₋₅acyl; R₅ₐ is H, monohalomethyl, C₁₋₄alkyl, C₁₋₄alkoxy-methyl, C₁₋₄alkyl-thiomethyl, hydroxyethyl, hydroxypropyl, phenyl, aralkyl, C₂₋₄alkenyl or -alkynyl; R₆ₐ is H or C₁₋₄alkyl; R₇ₐ is H, C₁₋₄alkyl or a residue of formula (a) as defined above, Xₐ is O, S, SO or SO₂; and nₐ is an integer of 1 to 4; or a pharmaceutically acceptable salt, hydrate, solvate, isomer or prodrug thereof; in the manufacture of a medicament for the treatment or prophylaxis of scLE and related autoimmune cutaneous conditions.

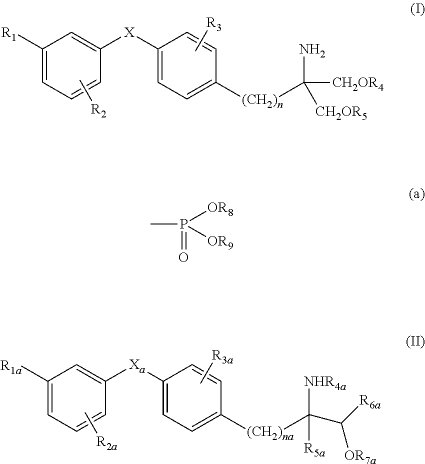

### FIELD OF THE INVENTION

The present invention relates to the treatment of Subacute Cutaneous Lupus Erythematosus (scLE) and related cutaneous autoimmune conditions.

### BACKGROUND OF THE INVENTION

ScLE is an autoimmune condition affecting the skin whose symptoms include symmetrical, non-scarring, erythematous, papulosquamous or annular lesions.

The pathology of scLE and related autoimmune cutaneous conditions is not well understood.

Symptoms can be triggered or worsened by exposure to UV light or as a side effect of taking medication for other conditions. Conventional first line agents for the treatment of scLE include antimalarials and locally or systemically applied steroids.

However, some patients do not respond to some or all of the above traditional treatments. In cases where patients do not respond to first line treatments and/or suffer adverse side effects, immunomosuppressant agents such as methotrexate or azathioprine are sometimes prescribed as a second line therapy. Alternative second/third line treatments include thalidomide. However, the use of these drugs is also not universally successful and is often associated with side effects such as increased susceptibility to opportunistic infection. Thalidomide also suffers from the side effect that it is neurotoxic. Therefore, there is a need for improved and/or alternative treatments for scLE and related cutaneous autoimmune conditions in order to expand the range of available therapies, particularly for the treatment of patients that are non-responsive to one or more of the traditional first and second line treatments or that experience adverse effects from these treatments.

### BRIEF DESCRIPTION OF THE INVENTION

In a First Aspect of the invention, there is provided the use of a compound of formula I:

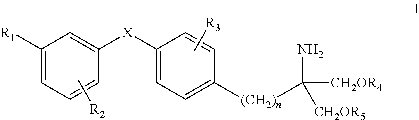

wherein X is O, S, SO or SO₂;
R₁ is halogen, trihalomethyl, —OH, C₁₋₇alkyl, C₁₋₄alkoxy, trifluoromethoxy, phenoxy, cyclohexylmethyloxy, pyridylmethoxy, cinnamyloxy, naphthylmethoxy, phenoxymethyl, —CH₂—OH, —CH₂—CH₂—OH, C₁₋₄alkylthio, C₁₋₄alkylsulfinyl, C₁₋₄alkylsulfonyl, benzylthio, acetyl, nitro or cyano, or phenyl, phenylC₁₋₄alkyl or phenyl-C₁₋₄alkoxy each phenyl group thereof being optionally substituted by halogen, CF₃, C₁₋₄alkyl or C₁₋₄alkoxy;
R₂ is H, halogen, trihalomethyl, C₁₋₄alkoxy, C₁₋₇alkyl, phenethyl or benzyloxy;
R₃H, halogen, CF₃, OH, C₁₋₇alkyl, C₁₋₁₄alkoxy, benzyloxy, phenyl or C₁₋₄alkoxymethyl;
each of R₄ and R₅, independently is H or a residue of formula (a)

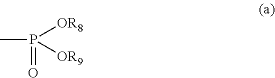

wherein each of R₈ and R₉, independently, is H or C₁₋₄alkyl optionally substituted by halogen;
and n is an integer from 1 to 4;
or a pharmaceutically acceptable salt, hydrate, solvate, isomer or prodrug thereof;
or a compound of formula II

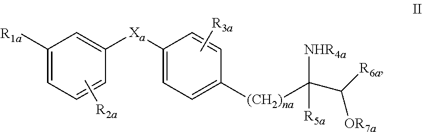

wherein
R₁ₐ is halogen, trihalomethyl, C₁₋₄alkyl, C₁₋₄alkoxy, C₁₋₄alkylthio, C₁₋₄alkylsulifinyl, C₁₋₄alkyl-sulfonyl, aralkyl, optionally substituted phenoxy or aralkyloxy;
R₂ₐ is H, halogen, trihalomethyl, C₁₋₄alkyl, C₁₋₄alkoxy, aralkyl or aralkyloxy;
R₃ₐ is H, halogen, CF₃, C₁₋₄alkyl, C₁₋₄alkoxy, C₁₋₄alkylthio or benzyloxy;
R₄ₐ is H, C₁₋₄alkyl, phenyl, optionally substituted benzyl or benzoyl, or lower aliphatic C₁₋₅acyl;
R₅ₐ is H, monohalomethyl, C₁₋₄alkyl, C₁₋₄alkoxy-methyl, C₁₋₄alkyl-thiomethyl, hydroxyethyl, hydroxypropyl, phenyl, aralkyl, C₂₋₄alkenyl or -alkynyl;
R₆ₐ is H or C₁₋₄alkyl;
R₇ₐ is H, C₁₋₄alkyl or a residue of formula (a) as defined above,
Xₐ is O, S, SO or SO₂; and
nₐ is an integer of 1 to 4;
   or a pharmaceutically acceptable salt, hydrate, solvate, isomer or prodrug thereof; in the manufacture of a medicament for the treatment or prophylaxis of scLE and related autoimmune cutaneous conditions.

In a Second Aspect of the invention, there is provided a compound of formula I or II as defined in the First Aspect of the invention or a pharmaceutically acceptable salt, hydrate, solvate, isomer or prodrug thereof, for use in a method for the treatment or prophylaxis of scLE and related autoimmune cutaneous conditions.

In a Third Aspect of the invention, there is provided a method of treating or preventing scLE and related autoimmune cutaneous conditions comprising administering to a subject in need thereof a therapeutically effective dose of a compound of formula I or II as defined in the First Aspect of the invention or a pharmaceutically acceptable salt, hydrate, solvate, isomer or prodrug thereof.

### DETAILED DESCRIPTION OF THE INVENTION

### Autoimmune Cutaneous Conditions

Autoimmune cutaneous conditions related to scLE include Acute Cutaneous Lupus Erythamatosus (acLE), Bullous Lupus Erythematosus (bLE), Chronic Cutaneous Lupus Erythamatosus (ccLE), Hypertrophic Lupus Erythematosus (hLE), Lupus Erythematosus Pannicilitis (LEp) and Lupus Erythematosus Tumidus (LEt).

### Patient Population

The compounds for use in the invention may be administered to patients as a first or second/third line therapy. In an aspect of the invention, the compounds of the invention are administered to patients as a first line therapy. In a further aspect of the invention, the compounds of the invention are administered to patients refractory to, or adversely affected by, traditional first line treatments e.g. antimalarials and/or locally or systemically applied steroids. In an aspect of the invention, the compounds of the invention are administered to patients refractory to, or adversely affected by, traditional second line treatments e.g. immunomosuppressant agents such as methotrexate or azathioprine or other second line treatments such as thalidomide.

### Compounds for Use in the Invention

With regard to the compounds of formulae (I) and (II), the term “halogen” encompasses fluorine, chlorine, bromine and iodine. The term “trihalomethyl” encompasses trifluoromethyl and trichloromethyl. “C₁₋₇ alkyl” encompasses straight-chained or branched alkyl, e.g. methyl, ethyl, propyl, isopropyl, butyl, t-butyl, pentyl, hexyl or heptyl. The phrase “optionally substituted phenoxy” encompasses unsubstituted phenoxy groups and those that have, at any position of its benzene ring, a halogen atom, such as fluorine, chlorine, bromine and iodine, trifluoromethyl, C₁₋₄alkyl or C₁₋₄alkoxy. The term “aralkyl” as in “aralkyl group” or “aralkyloxy group” encompasses benzyl, diphenylmethyl, phenethyl and phenylpropyl. Any C₁₋₄alkyl moiety e.g. as present in “C₁₋₄alkoxy”, “C₁₋₄alkylthio”, “C₁₋₄alkylsulfinyl” or “C₁₋₄alkylsulfonyl” encompasses straight-chained or branched C₁₋₄alkyl, e.g. methyl, ethyl, propyl, isopropyl or butyl. The phrase “optionally substituted aralkyl group” encompasses unsubstituted aralkyl groups and those that have, at any position of its benzene ring, a halogen atom, such as fluorine, chlorine, bromine and iodine, trifluoromethyl, lower alkyl having 1-4 carbon atoms, or lower alkoxy having 1-4 carbon atoms.

Preferred compounds of formula I are compounds of formula Ia

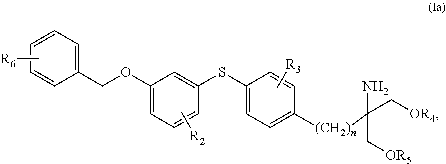

wherein
R₂, R₃, R₄, R₅ and n are as defined above; and
R₆ is hydrogen, halogen, C₁₋₇alkyl, C₁₋₄alkoxy or trifluoromethyl.

Further preferred compounds of formula (Ia) are those wherein R₃ is chlorine, e.g.

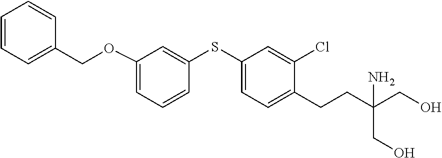 [SMILES: NC(CO)(CO)CCc1ccc(Sc2cccc(OCc3ccccc3)c2)cc1Cl]

2-amino-2-[4-(3-benzyloxyphenylthio)-2-chlorophenyl]ethyl-propane-1,3-diol
   and its corresponding phosphate derivative, phosphoric acid mono-2-amino-2-[4-(3-benzyloxyphenylthio)-2-chlorophenyl]ethyl-propyl]ester.

The phosphoric acid mono-2-amino-2-[4-(3-benzyloxyphenylthio)-2-chlorophenyl]ethyl-propyl]ester can be prepared enantiomerically pure by the procedures described in WO 2005/021503 to give:

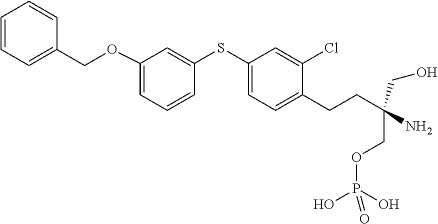 [SMILES: N[C@](CO)(CCc1ccc(Sc2cccc(OCc3ccccc3)c2)cc1Cl)COP(=O)(O)O]

Phosphoric acid mono-{(S)-2-amino-4-[4-(3-benzyloxy-phenylsulfanyl)-2-chloro-phenyl]-2-hydroxymethyl-butyl}ester
   or

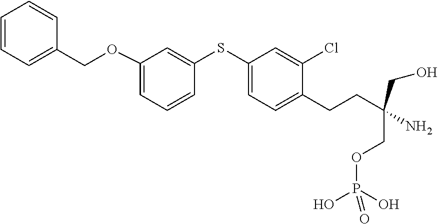 [SMILES: N[C@@](CO)(CCc1ccc(Sc2cccc(OCc3ccccc3)c2)cc1Cl)COP(=O)(O)O]

Phosphoric acid mono-{(R)-2-amino-4-[4-(3-benzyloxy-phenylsulfanyl)-2-chloro-phenyl]-2-hydroxymethyl-butyl}ester

Preferred compounds of formula II are compounds of formula (IIa)

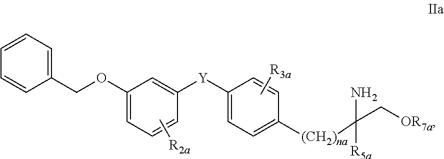

wherein
Y is O or S; and
   R₂ₐ, R₃ₐ, R₅ₐ, R₇ₐ and nₐ are as defined above.

Preferred compounds of formula (IIa) are those wherein R₃ is chlorine, e.g., 2-amino-4-[4-(3-benzyloxyphenylthio)-2-chlorophenyl]-2-methylbutane-1-ol; the corresponding phosphoric acid mono-2-amino-4-[4-(3-benzyloxyphenylthio)-2-chlorophenyl]-2-methylbutyl]ester; 2-amino-4-[4-(3-benzyloxyphenylthio)-2-chlorophenyl]-2-ethylbutane-1-ol; and the corresponding phosphoric acid mono-2-amino-4-[4-(3-benzyloxyphenylthio)-2-chlorophenyl]-2-ethylbutyl]ester.

Compounds of formulae I and II are known and are disclosed e.g. in WO03/029205, WO 03/029184 and WO04/026817, respectively, the phosphorylated derivatives being disclosed e.g. in WO04/074297, the contents of which being incorporated herein by reference in their entirety. Compounds of formulae I and II may be prepared as disclosed in above cited references.

Phosphorylated derivatives of compounds of formula (I), e.g., phosphoric acid mono-2-amino-2-[4-(3-benzyloxyphenylthio)-2-chlorophenyl]ethyl-propyl]ester, can be prepared utilizing the procedures for synthesizing phosphorylated compounds described e.g., in WO 2005/021503 (see, e.g., pages 11 and 12). Optically active compounds of structural formula (I) and phosphorylated derivatives thereof, in particular of formula (Ia) can be prepared in high purity utilizing the procedure described, e.g., in Hinterding et al., Synthesis, Vol. 11, pp. 1667-1670 (2003). As an example, an optically active compound of structural formula (Ia), phosphoric acid mono-2-amino-2-[4-(3-benzyloxyphenylthio)-2-chlorophenyl]ethyl-propyl]ester, can be prepared as described in the scheme below utilizing the procedures of Hinterding et al. (2003) supra.

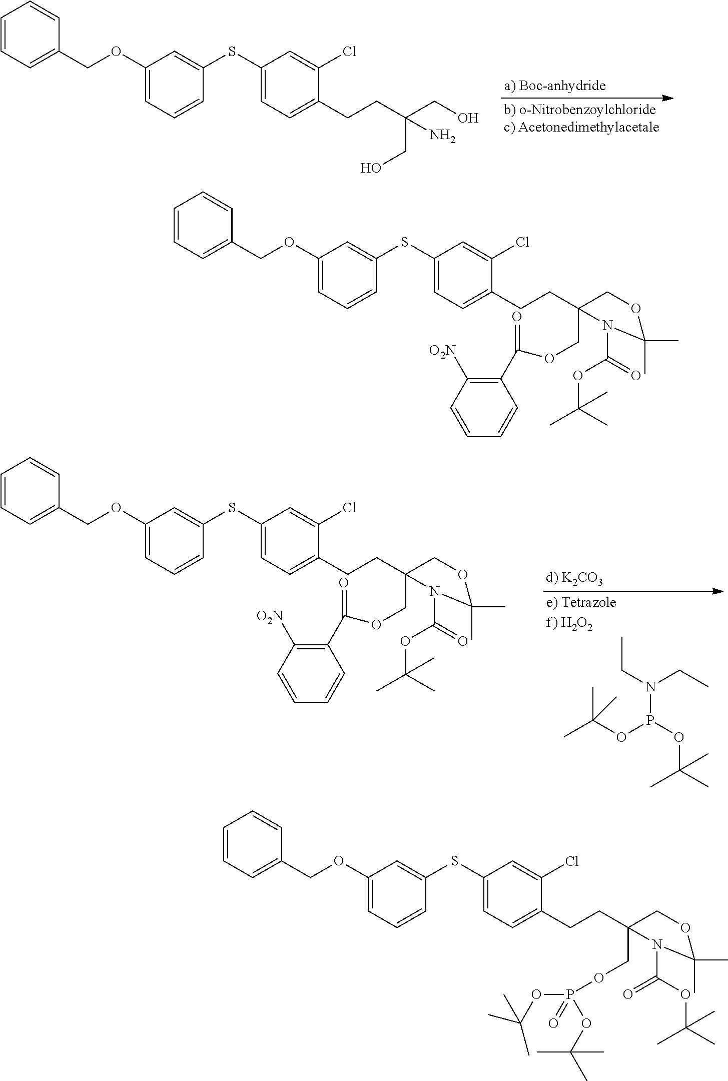

a) 1 equivalent of compound 1 and 1.2 equivalents Boc-anhydride in dioxane/acetonitrile or DMF/water (depends on solubility)+1.2 equivalents NaOH 1 M in water (RT, overnight).
b) 1 equivalent of step a), 1.5 equivalents 2-nitrobenzoylchloride and 1.6 equivalents pyridine in CH₂Cl₂ (RT, overnight).
c) 1 equivalent of step b), 3 equivalents acetonedimethylacetale and 0.1 equivalents p-TsOH.H₂O in toluene (95° C., 3 hours).
d) 1 equivalent of step c) and 0.075 equivalents K₂CO₃ (powder) in MeOH/THF (1/1) (RT, 4 hours).
e) 1 equivalent of step a), 6 equivalents tetrazole (recrystallized from toluene or 0.45 M in CH₃CN) and 2 equivalents di-t-butyldiethylphosphoramidite in dry THF (RT, 3 hours).
f) 5 equivalents H₂O₂ (30%) directly into the reaction mixture of step e) (0° C., 1 hour).

Isolation: the reaction mixture is quenched with sodium thiosulfate (saturated in water) and extracted with ethyl acetate (3×).

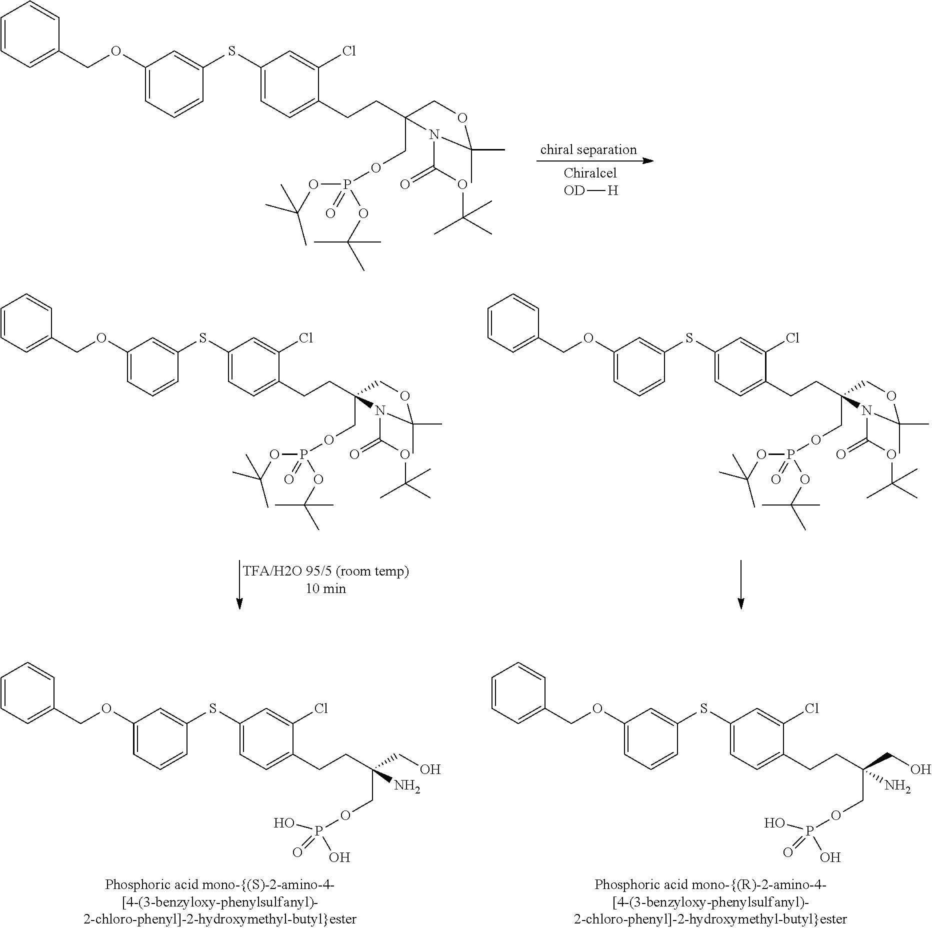

The compounds of formulae II and IIa, e.g., 2-amino-4-[4-(3-benzyloxyphenylthio)-2-chlorophenyl]-2-methylbutane-1-ol and 2-amino-4-[4-(3-benzyloxyphenylthio)-2-chlorophenyl]-2-ethylbutane-1-ol can be prepared as described e.g., in EP 1 548 003 A1. Preparation of such compounds of formulae II and IIa in high optical purity, can be prepared by the procedures described e.g., in Hinterding et al. (2003), supra; and Hinterding et al., Tetra Lett, Vol. 43, No. 45, pp. 8095-8097 (2002). Optically active phosphate derivatives of compounds of structural formulae II and IIa, e.g., phosphoric acid mono-2-amino-4-[4-(3-benzyloxyphenylthio)-2-chlorophenyl]-2-methylbutyl]ester and phosphoric acid mono-2-amino-4-[4-(3-benzyloxyphenylthio)-2-chlorophenyl]-2-ethylbutyl]ester can be prepared in high purity as described in Hinterding et al. (2003), supra.

The compounds of formulae I and II may exist in free form or salt form, or as a prodrug, solvate or hydrate.

Examples of pharmaceutically acceptable salts of the compounds of the formulae I and II include salts with inorganic acids, such as hydrochloride and hydrobromide salts and salts with organic acids, such as acetate, trifluoroacetate, citrate, tartrate and methanesulfonate salts.

When the compounds of formula I and II have one or more asymmetric centers in the molecule various optical isomers are obtained. The present invention embraces enantiomers, racemates, diastereoisomers and mixtures thereof. Moreover, when compounds of formula I and II include geometric isomers, the present invention embraces cis-compounds, trans-compounds and mixtures thereof.

The invention provides forms of the compound that have a hydroxyl or amine group present in a protected form; these function as prodrugs. Prodrugs are compounds that are converted into an active drug form after administration, through one or more chemical or biochemical transformations. Forms of the compounds of the present invention that are readily converted into the claimed compound under physiological conditions are prodrugs of the claimed compounds and are within the scope of the present invention. Examples of prodrugs include forms where a hydroxyl group is acylated to form a relatively labile ester such as an acetate ester, and forms where an amine group is acylated with the carboxylate group of glycine or an L-amino acid such as serine, forming an amide bond that is particularly susceptible to hydrolysis by common metabolic enzymes.

The term “effective amount” refers to an amount of a compound of formula I or II which, when administered to the patient, is effective to treat scLE or a related cutaneous autoimmune condition. “Treatment” includes a reduction of symptoms of the disease and/or their severity. Treatment efficacy may be evaluated using any indicators known in the art within the ability of one skilled in the art (e.g. a reduction in the Cutaneous LE Disease Area and Severity Index (CLASI) test value, for example decrease in CLASI≧50% (or ΔCLASI≧5) in moderately active disease (CLASI criteria described in Bonilla-Martinez et al. Arch Dermatol. 2008; 144:173).

The assessment of safety and side effects is within the ability of one skilled in the art and may include, for example, physical examinations, dermatologic examination, electrocardiograms (ECGs), Mobile Cardiac Outpatient Telemetry (MCOT), ophthalmic examinations, vital signs, standard clinical laboratory evaluations, hematology, blood chemistry, urinalysis, adverse event and serious adverse event monitoring.

“Prophylaxis” includes disease prevention or a reduction in disease recurrence.

Daily dosages required in practicing the method of the present invention will vary depending upon, for example, the compound used, the host, the mode of administration and the severity of the condition to be treated. A preferred daily dosage range is about from 0.1 to 100 mg as a single dose or in divided doses. Suitable daily dosages for patients are on the order of from e.g. 0.1 to 50 mg p.o. The compound may be administered by any conventional route, in particular enterally, e.g. orally, e.g. in the form of tablets, capsules, drink solutions, nasally, pulmonary (by inhalation) or parenterally, e.g. in the form of injectable solutions or suspensions. Suitable unit dosage forms for oral administration comprise from ca. 0.1 to 30 mg, usually 0.25 to 30 mg active ingredient, e.g. from about 0.1-5 mg, together with one or more pharmaceutically acceptable diluents or carriers therefore.

Compounds of formula I or II may be administered by any conventional route, in particular, enterally, e.g., orally, e.g., in the form of tablets or capsules, or parenterally, e.g., in the form of injectable solutions or suspensions, topically, e.g., in the form of lotions, gels, ointments or creams, or in a nasal or a suppository form. Phosphate derivatives of the compounds of formula I or II are preferably administered parenterally. Pharmaceutical compositions comprising such compounds in free form or in pharmaceutically acceptable salt form in association with at least one pharmaceutical acceptable carrier or diluent may be manufactured in conventional manner by mixing with a pharmaceutically acceptable carrier or diluent.

The compounds of formula I or II may be administered in free form or in pharmaceutically acceptable salt or prodrug form, e.g., as indicated above. Such salts may be prepared in conventional manner and exhibit the same order of activity as the free compounds.

The compounds of the invention give significant benefits compared to some or all of the prior art treatment methods. For example, the compounds do not exhibit the same general immunosuppressant activity as traditional second line treatment agents such as methotrexate or azathioprine thereby reducing the risk of opportunistic infection during treatment. Moreover, no neurotoxicity, a relatively common adverse effect of thalidomide, a further second/third line agent in refractory scLE, is expected with the use of the presently claimed compounds. In addition, the compounds of the invention are generally well tolerated by patients and may exhibit a favourable safety profile relative to some or all of the prior art treatment methods including e.g. cardiac safety (e.g. no or less pronounced heart rate reduction and/or AV blocks), renal safety (e.g. as measured by asymptotic elevation of liver enzymes) or pulmonary safety. In addition, treatment using the compounds of the invention may give rise to a reduction in other side effects observed in prior art methods (e.g dizziness, teratogenicity, nausea, fatigue, anemia, neuropenia, vomiting, increased risk of bruising, hair loss, constipation, deep vein thrombosis, atelactasis, refractory hypotension thinning of the skin, permanent dilation of certain blood vessels, burn marks on skin, liver and kidney damage and a weakened immune system) relative to some or all of the prior art treatment methods.

Utility of the compounds of formulae I and II in treating the diseases, disorders or conditions as hereinabove specified, may be demonstrated in clinical trials, for example in accordance with the methods hereinafter described.

### Clinical Trial

### Description of Trial

Efficacy of the compounds of formula I and II, (e.g. 2-amino-2-[4-(3-benzyloxyphenylthio)-2-chlorophenyl]ethyl-propane-1,3-diol) in treatment of scLE and related cutaneous autoimmune conditions may be tested in a randomised trial as follows.

Up to 24 18-75 year old patients with active scLE may be tested using 2-amino-2-[4-(3-benzyloxyphenylthio)-2-chlorophenyl]ethyl-propane-1,3-diol.

### Key Inclusion Criteria:

Diagnosis of SCLE (defined by Sontheimer et al, [Sontheimer R D, Thomas J R, Gilliam J N. Subacute cutaneous lupus erythematosus: a cutaneous marker for a distinct lupus erythematosus subset. Arch Dermatol 1979; 115:1409-15] including typical papulosquamous lannular skin lesions, positive anti-Ro antibody, photosensitivity, mild systemic involvement (e.g. arthralgia, arthritis, myalgia), positive biopsy); failure to respond to at least one standard therapy with steroids (topical or systemic) or antimalarials; active cutaneous lupus (as defined by CLASI≧6)

### Key Exclusion Criteria:

Pregnancy or lactation; any systemic immunosuppressive therapy within the last 4 weeks; any topical therapy within the last 2 weeks except the use of emollients; significant internal organ damage (e.g nephritis, CNS involvement).
   Concomitant Medication for scLE:
Only emollients allowed.

### Primary Endpoint

Change in Cutaneous LE Disease Area and Severity Index (CLASI), for example decrease in CLASI≧50% (or ΔCLASI≧5) in moderately active disease (CLASI criteria described in Bonilla-Martinez et al. Arch Dermatol. 2008; 144:173).

### Secondary Endpoints

Histological analyses of skin biopsies at Baseline and end of treatment (week 12) will assess the change in lymphocytic infiltration to serve as a Proof-of-Mechanism
Colorimetry (digital photography) to quantify the degree of lesional edema to confirm the results based on CLASI measurements.
Systemic Lupus Erythematosus Disease Activity Index (SLEDAI) will also be used.

### Treatment Period:

12 weeks

### Dose:

Once daily dosing to achieve a ˜70% reduction in peripheral ALC

### Data Collection

Clinical scores and lab data: at screening, weeks 0 (baseline), 2, 4 and 8 and 12
Biopsy: baseline and week 12

### Follow-up

for responders: 12 weeks and for non-responders: 4 weeks

### Sample Size:

enroll maximum of 24 patients (to have 20 available for analysis at end of study)

### SUMMARY OF THE INVENTION

Embodiment 1 relates to a compound of formula I or a pharmaceutically acceptable salt, hydrate, solvate, isomer or prodrug thereof, for use in the treatment or prophylaxis of scLE (Subacute Cutaneous Lupus Erythematosus) and related autoimmune cutaneous conditions:

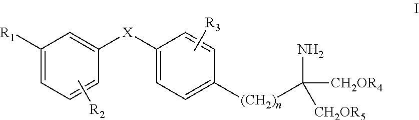

wherein X is O, S, SO or SO₂;
R₁ is halogen, trihalomethyl, —OH, C₁₋₇alkyl, C₁₋₄alkoxy, trifluoromethoxy, phenoxy, cyclohexylmethyloxy, pyridylmethoxy, cinnamyloxy, naphthylmethoxy, phenoxymethyl, —CH₂—OH, —CH₂—CH₂—OH, C₁₋₄alkylthio, C₁₋₄alkylsulfinyl, C₁₋₄alkylsulfonyl, benzylthio, acetyl, nitro or cyano, or phenyl, phenylC₁₋₄-alkyl or phenyl-C₁₋₄alkoxy each phenyl group thereof being optionally substituted by halogen, CF₃, C₁₋₄alkyl or C₁₋₄alkoxy;
R₂ is H, halogen, trihalomethyl, C₁₋₄alkoxy, C₁₋₇alkyl, phenethyl or benzyloxy;
R₃H, halogen, CF₃, OH, C₁₋₇alkyl, C₁₋₄alkoxy, benzyloxy, phenyl or C₁₋₄alkoxymethyl;
each of R₄ and R₅, independently is H or a residue of formula (a)

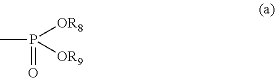

wherein each of R₈ and R₉, independently, is H or C₁₋₄alkyl optionally substituted by halogen;
and n is an integer from 1 to 4;
or a compound of formula II or a pharmaceutically acceptable salt, hydrate, solvate, isomer or prodrug thereof, for use in the treatment or prophylaxis of scLE and related autoimmune cutaneous conditions:

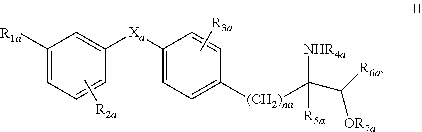

wherein
R₁ₐ is halogen, trihalomethyl, C₁₋₄alkyl, C₁₋₄alkoxy, C₁₋₄alkylthio, C₁₋₄alkylsulfinyl, C₁₋₄alkyl-sulfonyl, aralkyl, optionally substituted phenoxy or aralkyloxy;
R₂ₐ is H, halogen, trihalomethyl, C₁₋₄alkyl, C₁₋₄alkoxy, aralkyl or aralkyloxy;
R₃ₐ is H, halogen, CF₃, C₁₋₄alkyl, C₁₋₄alkoxy, C₁₋₄alkylthio or benzyloxy;
R₄ₐ is H, C₁₋₄alkyl, phenyl, optionally substituted benzyl or benzoyl, or lower aliphatic C₁₋₅acyl;
R₅ₐ is H, monohalomethyl, C₁₋₄alkyl, C₁₋₄alkoxy-methyl, C₁₋₄alkyl-thiomethyl, hydroxyethyl, hydroxypropyl, phenyl, aralkyl, C₂₋₄alkenyl or -alkynyl;
R₆ₐ is H or C₁₋₄alkyl;
R₇ₐ is H, C₁₋₄alkyl or a residue of formula (a) as defined above,
Xₐ is O, S, SO or SO₂; and
nₐ is an integer of 1 to 4.

Embodiment 2 relates to a compound for use according to embodiment 1, wherein the compound of formula I or II is, respectively, a compound of formula Ia

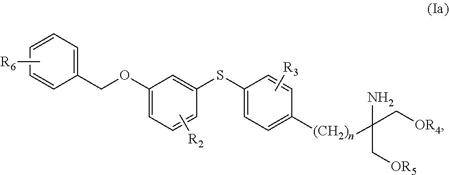

wherein
R₂, R₃, R₄, R₅ and n are as defined in claim 1; and
R₆ is hydrogen, halogen, C₁₋₇alkyl, C₁₋₄alkoxy or trifluoromethyl;
or a pharmaceutically acceptable salt, hydrate, solvate, isomer or prodrug thereof,
or a compound of formula (IIa)

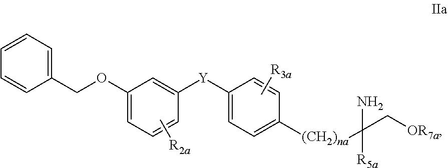

wherein
Y is O or S; and
R₂ₐ, R₃ₐ, R₅ₐ, R₇ₐ and nₐ are as defined in claim 1.
   or a pharmaceutically acceptable salt, hydrate, solvate, isomer or prodrug thereof.

Embodiment 3 relates to a compound for use according to any one of embodiment 1 or embodiment 2, wherein the compound of formula I is selected from:

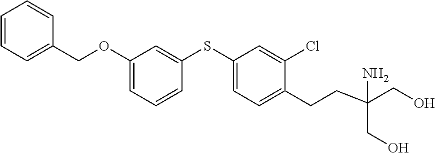 [SMILES: NC(CO)(CO)CCc1ccc(Sc2cccc(OCc3ccccc3)c2)cc1Cl]

2-amino-2-[4-(3-benzyloxyphenylthio)-2-chlorophenyl]ethyl-propane-1,3-diol
   or a pharmaceutically acceptable salt, hydrate, solvate, isomer or prodrug thereof,
   and its corresponding phosphate derivatives:

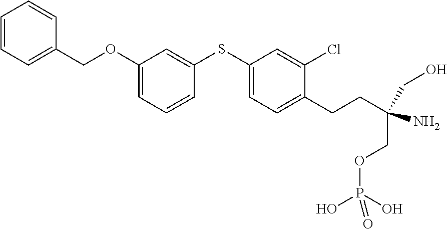 [SMILES: N[C@](CO)(CCc1ccc(Sc2cccc(OCc3ccccc3)c2)cc1Cl)COP(=O)(O)O]

Phosphoric acid mono-{(S)-2-amino-4-[4-(3-benzyloxy-phenylsulfanyl)-2-chloro-phenyl]-2-hydroxymethyl-butyl}ester,
   or a pharmaceutically acceptable salt, hydrate, solvate, isomer or prodrug thereof,
   or

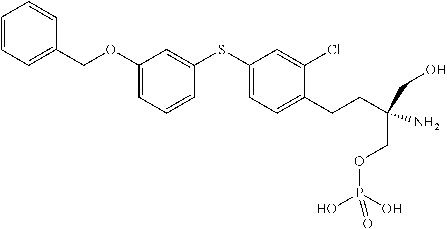 [SMILES: N[C@@](CO)(CCc1ccc(Sc2cccc(OCc3ccccc3)c2)cc1Cl)COP(=O)(O)O]

Phosphoric acid mono-{(R)-2-amino-4-[4-(3-benzyloxy-phenylsulfanyl)-2-chloro-phenyl]-2-hydroxymethyl-butyl}ester,
   or a pharmaceutically acceptable salt, hydrate, solvate, isomer or prodrug thereof.

Embodiment 4 relates to a compound for use according to any one of embodiments 1 to 3,

wherein the compound of formula I is selected from:

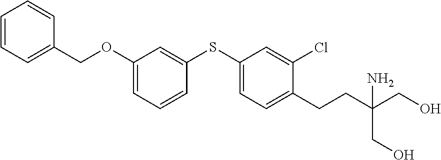 [SMILES: NC(CO)(CO)CCc1ccc(Sc2cccc(OCc3ccccc3)c2)cc1Cl]

2-amino-2-[4-(3-benzyloxyphenylthio)-2-chlorophenyl]ethyl-propane-1,3-diol
   or a pharmaceutically acceptable salt, hydrate, solvate, isomer or prodrug thereof.

Embodiment 5 relates to a compound for use according to any one of embodiments 1 to 4, wherein said treatment or prophylaxis is selected from scLE (Subacute Cutaneous Lupus Erythematosus), Acute Cutaneous Lupus Erythematosus, Bullous Lupus Erythematosus, Chronic Cutaneous Lupus Erythematosus, Hypertrophic Lupus Erythematosus, Lupus Erythematosus Pannicilitis, Lupus Erythematosus Tumidus and Neonatal Lupus Erythematosus.

Embodiment 6 relates to the use of compound of formula I or II as defined in any one of embodiments 1 to 4 or a pharmaceutically acceptable salt, hydrate, solvate, isomer or prodrug thereof, in the preparation of a medicament for the treatment or prophylaxis of scLE (Subacute Cutaneous Lupus Erythematosus) and related autoimmune cutaneous conditions.

Embodiment 7 relates to a method of treating or preventing scLE (Subacute Cutaneous Lupus Erythematosus) and related autoimmune cutaneous conditions comprising administering to a subject in need thereof a therapeutically effective amount of a compound of formula I or II as defined in any one of embodiments 1 to 4 or a pharmaceutically acceptable salt, hydrate, solvate, isomer or prodrug thereof.

Embodiment 8 relates to the use, the compound or the method of any one of embodiments 1 to 7, wherein the patient in need for treatment or prophylaxis is refractory to, or adversely affected by, traditional first and/or second line treatments for scLE and related conditions.

1. A method of treating or preventing scLE (Subacute Cutaneous Lupus Erythematosus) and related autoimmune cutaneous conditions comprising administering to a subject in need thereof a therapeutically effective amount of a compound of formula I: 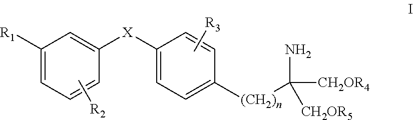
wherein X is O, S, SO or SO₂;
R₁ is halogen, trihalomethyl, —OH, C₁₋₇alkyl, C₁₋₄alkoxy, trifluoromethoxy, phenoxy, cyclohexylmethyloxy, pyridylmethoxy, cinnamyloxy, naphthylmethoxy, phenoxymethyl, —CH₂—OH, —CH₂—CH₂—OH, C₁₋₄alkylthio, C₁₋₄alkylsulfinyl, C₁₋₄alkylsulfonyl, benzylthio, acetyl, nitro or cyano, or phenyl, phenylC₁₋₄alkyl or phenyl-C₁₋₄alkoxy each phenyl group thereof being optionally substituted by halogen, CF₃, C₁₋₄alkyl or C₁₋₄alkoxy;
R₂ is H, halogen, trihalomethyl, C₁₋₄alkoxy, C₁₋₇alkyl, phenethyl or benzyloxy;
R₃H, halogen, CF₃, OH, C₁₋₇alkyl, C₁₋₄alkoxy, benzyloxy, phenyl or C₁₋₄alkoxymethyl;
each of R₄ and R₅, independently is H or a residue of formula (a) 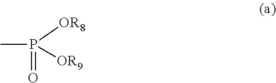
wherein each of R₈ and R₉, independently, is H or C₁₋₄alkyl optionally substituted by halogen;
and n is an integer from 1 to 4; or a pharmaceutically acceptable salt, hydrate, solvate, isomer or prodrug thereof;
or a compound of formula II: 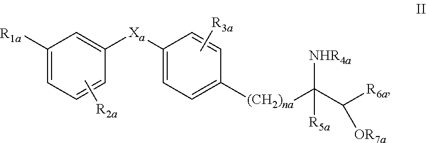
wherein
R₁ₐ is halogen, trihalomethyl, C₁₋₄alkyl, C₁₋₄alkoxy, C₁₋₄alkylthio, C₁₋₄alkylsulfinyl, C₁₋₄alkyl-sulfonyl, aralkyl, optionally substituted phenoxy or aralkyloxy;
R₂ₐ is H, halogen, trihalomethyl, C₁₋₄alkyl, C₁₋₄alkoxy, aralkyl or aralkyloxy;
R₃ₐ is H, halogen, CF₃, C₁₋₄alkyl, C₁₋₄alkoxy, C₁₋₄alkylthio or benzyloxy;
R₄ₐ is H, C₁₋₄alkyl, phenyl, optionally substituted benzyl or benzoyl, or lower aliphatic C₁₋₅acyl;
R₅ₐ is H, monohalomethyl, C₁₋₄alkyl, C₁₋₄alkoxy-methyl, C₁₋₄alkyl-thiomethyl, hydroxyethyl, hydroxypropyl, phenyl, aralkyl, C₂₋₄alkenyl or -alkynyl;
R₆ₐ is H or C₁₋₄alkyl;
R₇ₐ is H, C₁₋₄alkyl or a residue of formula (a) as defined above,
Xₐ is O, S, SO or SO₂; and
nₐ is an integer of 1 to 4; or a pharmaceutically acceptable salt, hydrate, solvate, isomer or prodrug thereof.

2. The method according to claim 1, wherein the compound of formula I or II is, respectively, a compound of formula Ia 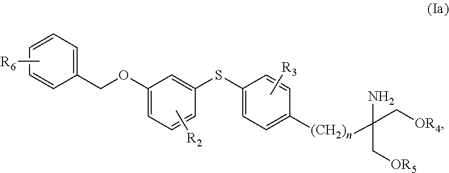
wherein
R₂, R₃, R₄, R₅ and n are as defined in claim 1; and
R₆ is hydrogen, halogen, C₁₋₇alkyl, C₁₋₄alkoxy or trifluoromethyl;
or a pharmaceutically acceptable salt, hydrate, solvate, isomer or prodrug thereof,
or a compound of formula (IIa) 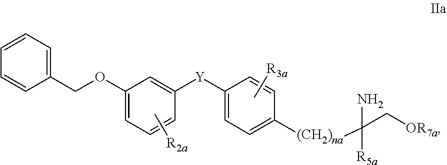
wherein
Y is O or S; and
R₂ₐ, R₃ₐ, R₅ₐ, R₇ₐ and nₐ are as defined in claim 1.
or a pharmaceutically acceptable salt, hydrate, solvate, isomer or prodrug thereof.

3. The method according to claim 1, wherein the compound of formula I is selected from: 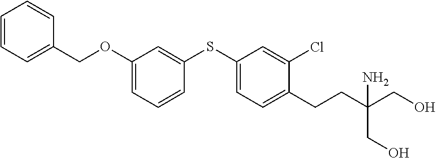 [SMILES: NC(CO)(CO)CCc1ccc(Sc2cccc(OCc3ccccc3)c2)cc1Cl]
2-amino-2-[4-(3-benzyloxyphenylthio)-2-chlorophenyl]ethyl-propane-1,3-diol
or a pharmaceutically acceptable salt, hydrate, solvate, isomer or prodrug thereof,
and its corresponding phosphate derivatives: 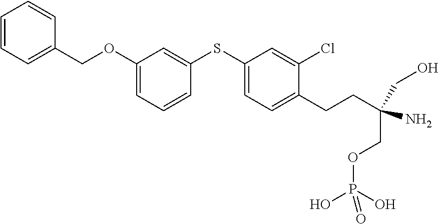 [SMILES: N[C@](CO)(CCc1ccc(Sc2cccc(OCc3ccccc3)c2)cc1Cl)COP(=O)(O)O]
Phosphoric acid mono-{(S)-2-amino-4-[4-(3-benzyloxy-phenylsulfanyl)-2-chloro-phenyl]-2-hydroxymethyl-butyl}ester,
or a pharmaceutically acceptable salt, hydrate, solvate, isomer or prodrug thereof,
or 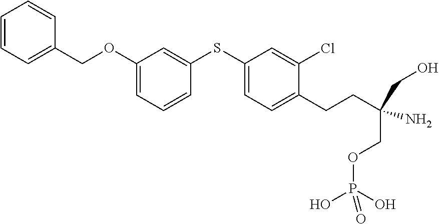 [SMILES: N[C@@](CO)(CCc1ccc(Sc2cccc(OCc3ccccc3)c2)cc1Cl)COP(=O)(O)O]
Phosphoric acid mono-{(R)-2-amino-4-[4-(3-benzyloxy-phenylsulfanyl)-2-chloro-phenyl]-2-hydroxymethyl-butyl}ester,
or a pharmaceutically acceptable salt, hydrate, solvate, isomer or prodrug thereof.

4. The method according to claim 1, wherein the compound of formula I is selected from: 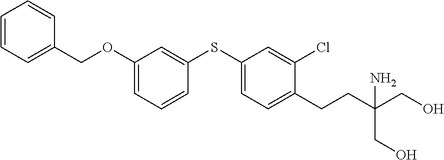 [SMILES: NC(CO)(CO)CCc1ccc(Sc2cccc(OCc3ccccc3)c2)cc1Cl]
2-amino-2-[4-(3-benzyloxyphenylthio)-2-chlorophenyl]ethyl-propane-1,3-diol
or a pharmaceutically acceptable salt, hydrate, solvate, isomer or prodrug thereof.

5. The method according to claim 1, wherein said treatment or prophylaxis is selected from scLE (Subacute Cutaneous Lupus Erythematosus), Acute Cutaneous Lupus Erythematosus, Bullous Lupus Erythematosus, Chronic Cutaneous Lupus Erythematosus, Hypertrophic Lupus Erythematosus, Lupus Erythematosus Pannicilitis, Lupus Erythematosus Tumidus and Neonatal Lupus Erythematosus.

6-8. (canceled)
